(19) United States
(12) Patent Application Publication
(10) Pub. No.: US 2023/0390495 A1
(43) Pub. Date: Dec. 7, 2023

(54) Nested Molded Piston Parts For Multi-Chamber Syringes

(71) Applicant: Thome Intellectual Property Holdings, LLC, Bountiful, UT (US)

(72) Inventors: Gale Harrison Thorne, JR., Bountiful, UT (US); Gale Harrison Thorne, Bountiful, UT (US)

(73) Assignee: Thome Intellectual Property Holdings, LLC, Bountiful, UT (US)

(21) Appl. No.: 17/803,552

(22) Filed: Aug. 24, 2022

Related U.S. Application Data
(63) Continuation-in-part of application No. 17/803,379, filed on Jun. 6, 2022.

(51) Int. Cl. A61M 5/315; A61M 5/31; A61M 5/19

(52) U.S. Cl. CPC A61M 5/31511; A61M 5/3134; A61M 5/3145; A61M 5/19; A61M 2005/3128; A61M 2005/1787

(57) ABSTRACT

Nested piston apparatus is disclosed for making free pistons used to provide multi-chamber mixing and sequential delivery syringes within conventional syringe barrels. Each such syringe comprising one or more free pistons made according to the instant invention whereby syringe contents are selectively kept disparate until acted upon by forced displacement of an associated plunger rod. As well, nested piston apparatus is disclosed for using nested piston apparatus as multiple free pistons for making triple chamber syringes.

### CONTINUATION-IN-PART

This Patent Application is a CONTINUATION-IN-PART of U.S. patent application Ser. No. 17/803,379, LIMITED STEP METHODS FOR MEDICINE STERILIZATION AND BOTTLING submitted by Gale H. Thorne, Jr. et al.

### FIELD OF INVENTION

The field of this invention concerns piston valves used in multi-chamber syringes, especially pistons which are employed for syringes used in medicine which are constructed using conventional barrels which are substantially hollow, linear cylinders and without grooves or other wall deformities.

### DESCRIPTION OF RELATED ART

Contemporary multi-chamber syringes are most commonly dual chamber syringes wherein a valved piston keeps matter within adjoining chambers disparate until a predetermined action which results in valve opening. In medicine, applications for multi-chamber syringes comprise syringes for sequential dose delivery with flush, mixing, mixing with flush and triple chamber syringes for flushing after sequential delivery.

An example of related art is U.S. Pat. No. 7,048,720, issued May 23, 2006, wherein a dome-shaped slit valve is activated to an open state by an increased pressure gradient across the valve, after a forward chamber is emptied. Valve actuation results in rear chamber delivery. To assure that no air is delivered from a back chamber, which cannot be purged at time of dispensing, a specially designed air-flow opposing filter part is disposed in a pathway between the rear and forward chambers. To assure that no liquid is displaced from the forward chamber into the rear chamber when a flow control piston and associated plunger rod are displaced proximally, a “stent” is provided as an inherent part of filter part construction.

Another example of related art is found in U.S. Pat. No. 7,789,862 issued Sep. 5, 2006, wherein a valve stem is displaced to open a valve for flow from an otherwise disparate rear chamber for dispensing. To assure no air is dispensed from the rear chamber, a flow path is provided from the rear chamber through a cylindrical channel disposed between a “liquid-only” zone in the rear chamber and the valve.

In both examples cited supra, only a single molded part is used for the valve piston. In the case of the first example, gas leakage between the filter part and dome due to variances in material characteristics between the filter and dome construction raised questions concerning reliability of operation which, for implementation, required special design and construction. Cost of the filter part, itself, was also of concern.

In a second example, a fluid flow path was required to provide, not only for liquid passage from the liquid-only zone, but also for displacement of the stem into the valve body. Such a requirement led to a larger than desired pathway diameter, reducing the effective volume of air which could be confined in the rear chamber.

A mixing syringe disclosed in U.S. Pat. No. 7,101,354, issued Sep. 5, 2006, disclosed using a single piston valve disposed between two chambers holding matter to be mixed. The valve was constructed as a one-way slit valve being conducive to flow from a rear chamber to a forward chamber while being obstructive otherwise. Mixing was effected by a volume of gas (air) disposed in the capped forward chamber such that as the piston valve is displaced forward, by displacing a rear piston and plunger rod against fluid in the rear chamber, liquid is displaced into the forward chamber and pressure is increased therein. When the force on the plunger rod is relieved, the increased pressure displaces the piston valve and remaining liquid in the rear chamber proximally. In this manner, all liquid can be displaced from the rear chamber into the forward chamber for mixing. The problem with this mixing device is that any inadvertent forward displacement could cause premature mixing which makes use of this device commercially inadvisable.

### DEFINITION OF TERMS

Following is a glossary of terms with specific associated definitions which are applicable to contents of this specification for the instant invention:
at least one, n: one or more
catch ring, n: a female trough in an interior face of a piston disposed along an interface with another piston having a latch ring which is inserted into the trough to join and nest the pistons.
conventional medical syringe, adj: ordinary or commonplace in construction, especially having a syringe barrel which is cylindrical, of nearly constant diameter and without grooves, slots or offsets of any kind where fluid displacement occurs therein.
distal, adj: away from a plunger piston and associated piston rod.
free piston, n: a syringe piston disposed within the barrel of a syringe which is not connected to a plunger piston and is therefore displaced by differential fluid pressure disposed upon opposing faces of the piston.
latch ring, n: a raised ring affixed to an interior face of a piston disposed along an interface with another piston having a female (catch) ring into which the raised ring is inserted to join and nest the pistons.
Liquid only zone, n: a space within a barrel of a syringe in which no gas can reside such that only liquid can be acquired to be accessed therefrom.
nested piston apparatus, n: at least two pistons nested together to form a free piston, at least one of the pistons having a valve which is closed to flow through this free piston until being selectively opened, this free piston further having a plurality of wiping rings, for engagement with a syringe barrel, (at least one ring being disposed about a most proximal piston and at least one other ring being disposed about a most distal piston) such that space between the wiping rings is disparate from space through which valve gated fluid flows.
nested, adj.: joining of two disparate parts to form an apparatus which operates as a single part, as an example, nested pistons within the barrel of a syringe.
proximal, adj: next to or nearer a plunger piston and associated piston rod.
piston, n: a device which is sized and structured to be disposed within a barrel of a syringe and used to displace fluid resident in the barrel when displaced therein.
piston rod, n: an elongated structure sized and shaped to provide for digital force to be applied to a piston rod piston to displace fluid within a barrel of an associated syringe.
piston rod piston, n: a device which is affixed to a piston rod and disposed within the barrel of a syringe, this piston being sized and shaped to act as a plunger for wiping fluid from the inner surface of the barrel such that when the piston rod is displaced within the barrel fluid within the barrel is also displaced.
SAL (Sterilization assurance level), n: a measurement of sterility.
substantially, adv: made within contemporary manufacturing capability of an item so specified.
wiping ring, n: a circumferential ring about a piston which is sized and shaped to wipe fluid from the inner surface of an interfacing syringe barrel as the piston is displaced. According to the instant invention each most distal and most proximal piston of a nested piston apparatus shall have at least one wiping ring affixed thereto.

### BRIEF SUMMARY AND OBJECTS OF THE INVENTION

In brief summary, this novel invention alleviates currently recited problems in multi-chamber syringes recited supra and all other known problems related to making and using both mixing and sequential delivery syringes. A plurality of instant inventions are hereafter disclosed in two stages, the first stage being dedicated to modes of construction of nested pistons. The second stage is related to definitive designs for making and using nested valved pistons for specific applications. In all cases, nested valves comprise structure and function which cannot be achieved in a singly molded part. Also, each exteriorly disposed part, of each piston which is nested with another part to form a nested syringe piston, comprises a cylindrical body which has at least one wiping ring.

### Stage 1—Nested Pistons

According to the instant invention, the term “nested pistons” is defined as a coupling of at least two free syringe piston parts, which remain together in a fluid tight relationship within a syringe barrel to provide a plurality of functions not possible by a single molded part. To “nest” two parts within a syringe body, the parts should have interfaces (joining part-to-part surfaces) which are substantially fluid tight and do not separate as the nested piston is displaced within a syringe barrel. It should be noted under conditions of free pistons within a syringe barrel, forced displacement always acts to keep the parts together.

Part joining can be accomplished in various ways, two examples of which are (1) the use of a matching and coupling interface between nesting part joining surfaces and (2) by means of conforming coupling rings and grooves in adjoining piston interfaces. Other means for joining may be used within the scope of the instant invention but are not addressed herein.

Generally, nested pistons according to the instant invention are for use in conventional syringes. Each of such syringes have a hollow cylindrically shaped barrel which is closed about a distally disposed fluid communicating spout with spout flow being curtailed by a removable cap. The barrel has a proximally disposed orifice for insertion of a plunger, which is sized and shaped to effectively displace fluid disposed within the barrel. In a conventional syringe, the plunger is inserted through the orifice to form a single distal chamber within the barrel. Each single chamber, within the scope of the invention, can be divided into two chambers by insertion of a free piston.

Nesting pistons provide an apparatus for constructing nested valves which provide a plurality of valve functions within each free piston. A plunger rod, associated with a piston which closes the proximal chamber, is generally employed for displacing free pistons and chamber disposed fluids within the barrel.

Stage 1—Piston and Valve structures

Example 1: A free nested piston apparatus which has a first piston having a collision sensitive valve disposed on the distal side of the apparatus and a second piston having an arcuate proximal surface having a medially disposed apex and a pathway to the first valve such that only liquid from a chamber proximal to the apparatus can be dispensed through the valve. This valve is effective in opening a flow path from a chamber proximal to the apparatus when the apparatus collides with the spout end of a syringe thereby permitting sequential dispensing of the proximal chamber after the distal chamber is emptied.

Example 2: A free nested piston apparatus which has a first piston comprising a one-way valve which is permissive to distally directed flow and a second piston having a pressure actuated valve which opens to permit distally directed flow only when actuated by a predetermined level of applied pressure. The two valves enclose an otherwise empty space within the piston apparatus which comprises a compressible fluid (e.g. air) permitting a pressure differential to be applied across the pressure actuated valve by applying distally directed force upon an associated plunger rod. When the nested piston is disposed within a syringe to provide a proximal and a distal chamber and both valves are closed, contents of distal and proximal chambers about the apparatus are kept disparate. When the pressure valve is actuated, fluid flow is permitted from the proximal chamber into the distal chamber.

Example 3: A free nested piston apparatus like the apparatus disclosed in Example 2, except for a sterilizing filter piston being disposed between the first and second pistons. The sterilizing filter acts to sterilize fluid dispensed through the apparatus (into the distal chamber).

Example 4: A free nested piston apparatus which has a dome valve disposed as a distal piston and a proximally disposed piston having geometry which keeps a slit in the dome valve closed until the dome is inverted resulting from reduction in downstream pressure and piston rod applied positive pressure. Further, the proximal piston has geometry which provides a hole which only has access to a liquid only zone as disclosed in U.S. Pat. No. 7,789,862 to assure no air is delivered through this apparatus. It may be noted that this apparatus is similar in function to the apparatus of Example 1, except that valve opening occurs due to a pressure gradient across the valve rather than opening due to collision.

Example 5: A free nested piston apparatus which has a distally disposed piston like the valve piston of Example 1, but in place of the liquid only zone filter, is a filter plunger similar to the sterilizing filter plunger of Example 3. In this case, the sterilizing filter plunger not only filters liquid being displaced there through but, also, impedes gas (air) flow as sterilizing filters, once wet, are impervious to gas.

### Stage 2—Nested Piston Applications

Application Example 1: Disposing the free nested piston apparatus of Example 1, to create a proximal and distal chamber within a syringe barrel. Fill the proximal chamber with a second-to-be dispensed liquid and the distal chamber and nested piston with a first-to-be dispensed liquid. Close the rear opening of the syringe with a plunger piston and rod. This construction yields a sequential delivery syringe having the following attributes:
a. Solutions in the two chambers are kept disparate.
b. A conventional medical syringe is employed.
c. Priming of the distal chamber is performed in the same manner as used in priming a single chamber syringe.
d. All gas is retained in the proximal chamber upon liquid being dispensed therefrom.
e. Contents of the syringe are dispensed disparately and sequentially.

Application Example 2: Use the free nested piston apparatus of Example 2, to create a mixing syringe within a conventional syringe barrel. Dispose the apparatus to provide a distal and a proximal chamber. In the distal chamber dispose matter to be mixed. Note that such matter may be solid, such as lyophilized powder or a liquid to be diluted and mixed with a second drug or diluent provided in the proximal chamber. Displacement of diluent (proximal chamber liquid) occurs when pressure is applied to a piston rod to open the proximal valve of the apparatus and discharge a portion of the proximal chamber liquid into the distal chamber. This action increases pressure in the distal chamber such that when force is relieved from the piston rod, the apparatus is displaced proximally. Repeated application of applying pressure upon the piston rod will sequentially “pump” all of the liquid from the proximal chamber into the distal chamber where mixing occurs. Features of such mixing are:
a. Solutions in the two chambers are kept disparate until a predetermined pressure is applied to an associated piston rod to open one of the nested piston valves.
b. A conventional medical syringe is employed.
c. Priming of the distal chamber is performed in the same manner as used in priming a single chamber syringe.
d. With sequential pumping, the entire contents of the proximal chamber can be displaced into the distal chamber to be mixed with matter therein.
e. No solution from the proximal chamber can be dispensed into the distal chamber until a pressure sufficient to open the proximal valve is applied within the barrel of the syringe.

Application Example 3: Use the free nested piston apparatus of Example 3 apparatus. Follow steps provided for Application Example 2. The basic difference between Application Example 2 and Application Example 3 is the sterilization of contents dispensed from the proximal chamber which are sterilized to a predetermined SAL by being displaced through a sterilizing filter. Thus, should there be concern for contamination either via production or time in chamber, this application provides a novel solution using nested pistons.

Application Example 4: Novelty of nested pistons may be best demonstrated by this application example which includes mixing and dispensing contents of two chambers and thereafter delivering a flush from a third chamber, all provided within a single conventional syringe. To provide a mixing syringe with flush, a nested piston apparatus of valve structure disclosed in example 2 of stage 1 is displaced into a syringe barrel to provide a distal and proximal chamber and a nested piston apparatus of valve structure of stage 1, example 4, is displaced into the barrel to divide the proximal chamber into a middle chamber and a most proximal chamber. Thus constructed, and in general, a three chamber syringe is provided which keeps each chamber disparate until a valve is actuated in sequence from distal to proximal chambers.

Accordingly, it is a primary object to provide nested free pistons for multi-chamber syringes for which no piston can be made as a single molded part.

It is a principle object to provide for part nesting such that valve parts are joined together (nested) in fluid tight relationship when disposed in a syringe barrel.

It is a fundamental object to provide nested valves which are selectively actuated within syringe barrels by either contact with an interior surface within a barrel or by differential pressure disposed about a valve.

It is a basic object to provide a nested piston valve which is effective in providing a mixing syringe.

It is another basic object to provide nested piston valves which are effective in providing sequential delivery syringes.

It is still another basic object to provide combinations of nested piston valves which are effective in providing nested piston valves for a mixing syringe with flush.

It is yet another basic object to provide combinations of nested piston valve for triple chamber sequential delivery syringes.

It is still another basic object to provide a nested valve for a mixing syringe which contains a sterilizing filter for sterilizing proximal chamber disposed liquid before contact with matter in a distal chamber.

These and other objects and features of the present invention will be apparent from the detailed description taken with reference to accompanying drawings.

### BRIEF DESCRIPTION OF THE DRAWINGS

FIG. 1 is a cross section of a PRIOR ART conventional medical syringe seen with a single chamber comprising fluid contents also seen in cross section.

FIG. 2 is a cross section of a conventional medical syringe like the syringe seen in FIG. 1 with a nested piston apparatus disposed within a barrel of the syringe to divide the single chamber into two disparate chambers.

FIG. 3 is a cross section of a conventional medical syringe like the syringe seen in FIG. 1 with two nested piston apparatuses disposed within a barrel of the syringe to divide the single chamber into three disparate chambers.

FIG. 4 is a side elevation of a piston used as a part of a nested piston apparatus.

FIG. 5 is a side elevation of a plug used within a free piston of a nested piston apparatus.

FIG. 6 is a side elevation of a piston used as a part of a nested piston apparatus.

FIG. 7 is a cross section of the piston seen in FIG. 4.

FIG. 8 is a cross section of the plug seen in FIG. 5.

FIG. 8A is a magnified view of a proximal side elevation of the plug seen in FIG. 8.

FIG. 9 is a cross section, as molded, of the piston seen in FIG. 6.

FIG. 10 is a frontal elevation of distal face of the piston seen in FIG. 9.

FIG. 11 is a side elevation of a nested piston apparatus assembled from parts seen in FIGS. 4, 5, and 6.

FIG. 12 is a cross section of the nested piston apparatus seen in FIG. 11.

FIG. 13 is a cross section of the nested piston apparatus seen in FIG. 12 with a valve portion having been actuated.

FIG. 14 is a side elevation of the nested piston apparatus seen in FIG. 13.

FIG. 15 is a cross section of a nested piston apparatus seen in FIG. 11 disposed as a free piston within a barrel of a syringe to divide the barrel into two chambers.

FIG. 15A is a cross section of the syringe and free piston seen in FIG. 15 with the nested piston apparatus seen in cross section disposed to keep fluid in a proximal chamber disparate from fluid disposed in a distal chamber.

FIG. 16 is a side elevation of a plunger rod.

FIG. 17 is a cross section of a syringe and nested piston apparatus seen in FIG. 15A with the free piston displaced and actuated by plunger rod (seen in FIG. 16) disposed within the barrel of the syringe seen in FIG. 15 having displaced and actuated the free nested piston apparatus whereby liquid has been dispensed from the distal chamber.

FIG. 18 is a cross section of a syringe with a proximal chamber of a barrel emptied by displacement of the plunger rod.

FIG. 19 is an exploded side elevation of two pistons made according to the instant invention for being joined (nested) to provide a nested piston apparatus.

FIG. 19A is an exploded view cross section of pistons seen in FIG. 19.

FIG. 20 is a frontal elevation of the proximal face of the nested piston apparatus wherein a one-way slit valve is seen.

FIG. 21 is a side elevation to the two parts seen in FIG. 19 nested to form a nested piston apparatus.

FIG. 21A is a cross section of the nested piston apparatus seen in FIG. 21.

FIG. 22 is a side elevation of the nested piston apparatus with a bulging portion of the apparatus, seen in FIG. 21, hidden from view as a result of actuation of a valve.

FIG. 22A is a cross section of the nested piston apparatus seen in FIG. 22.

FIG. 23 is a cross section of a nested piston apparatus, seen in FIG. 21A, and a piston rod and piston seen in FIG. 1 disposed within the syringe barrel and the cap, also seen in FIG. 1, which thereby provide a closed proximal chamber in which liquid is disposed and a distal chamber in which matter, such as a lyophilized powder, is disposed.

FIG. 24 is a cross section of parts seen in FIG. 23 with the piston rod piston displaced to open a valve of the nested piston apparatus.

FIG. 25 is a cross section wherein contents of the proximal chamber are displaced into the distal chamber of the syringe.

FIG. 26 is a cross section of a valved piston of a nested piston apparatus made according to the instant invention.

FIG. 27 is a cross section of a filter piston.

FIG. 28 is a cross section of a valved portion made to be nested with the parts seen in FIGS. 22 and 23.

FIG. 29 is a cross section of a nested piston apparatus which comprises parts seen in FIGS. 26-28.

FIG. 30 is a side elevation of the nested piston apparatus seen in FIG. 29.

FIG. 31 is a cross section of the nested piston apparatus seen in FIG. 29 with a valve of the part seen in FIG. 26 actuated to an open state.

FIG. 32 is a cross section of a syringe barrel (seen for example in FIG. 1) with the nested piston apparatus seen FIG. 29 disposed therein to provide a provide a proximal and a distal chamber.

FIG. 33 is a cross section similar to FIG. 32 with fluid seen disposed in proximal and distal chambers and a piston rod and plunger also disposed in the barrel for fluid containment.

FIG. 34 provides a cross section of the nested piston apparatus disposed within the barrel seen in FIG. 33 and the piston rod and plunger displaced to open a valve of the nested piston apparatus.

FIG. 35 provides a cross section of the nested piston apparatus, seen in FIG. 34, disposed proximally such that the fluid previously disposed in the proximal chamber is displaced into the distal chamber for mixing.

FIG. 36 is a cross section of a piston for nesting with a piston seen in FIG. 37 for providing a nested piston apparatus for separating a proximal chamber of a dual chamber syringe into a more proximal and a more distal chamber.

FIG. 37 is a cross section of a piston to be nested with the piston seen in FIG. 36.

FIG. 38 is a cross section showing a nested piston apparatus resulting by joining pistons seen in FIGS. 36 and 37.

FIG. 39 is a side elevation of the nested piston apparatus seen in FIG. 38.

FIG. 40 is a cross section of the nested piston apparatus seen in FIG. 38 with a valve actuated to an open state.

FIG. 41 is a cross section of two nested piston apparatuses disposed in a syringe to provide a three-chamber syringe comprising a proximal, a medial and a distal chamber, each chamber comprising liquid.

FIG. 42 comprises cross sections and parts similar to that of FIG. 41 with a valve in the distal nested piston apparatus being in an open state.

FIG. 43 comprises cross sections and parts similar to that of FIG. 42 with liquid in the medial chamber dispensed into the distal chamber.

FIG. 43A is a rotated view of a syringe seen in FIG. 43 rotated to an upright state with a cap removed therefrom.

FIG. 44 comprises valve cross sections similar to that of FIG. 43 with liquid dispensed from the distal chamber.

FIG. 45 comprises cross sections similar to that of FIG. 43 with a valve, in the proximally disposed nested piston apparatus, opened.

FIG. 46 comprises cross sections and parts similar to that of FIG. 45 with liquid in the proximal chamber dispensed.

FIG. 47 is a cross-section of a piston with a one-way valve disposed in a fluid path of the piston.

FIG. 48 is a frontal elevation of the piston seen in FIG. 47.

FIG. 49 is an exploded view of a nested piston apparatus comprising the piston seen in FIG. 47 and an offsetting piston.

FIG. 50 is an exploded view of a nested piston apparatus comprising a portion of the nested piston apparatus seen in FIG. 12 and an interfacing piston.

FIG. 51 is a cross section of the nested pistons seen in FIG. 50 joined together.

FIG. 52 is a cross section of the pistons seen in FIGS. 49 and 50 adjoined for insertion into a barrel of a syringe.

FIG. 53 is an exploded view of parts used to assembly a multi-chamber syringe according to the instant invention.

FIG. 54 is a cross section of parts, seen (except for a cap) in FIG. 53, assembled to make a multi-chamber syringe according to the instant invention.

FIG. 55 is cross section like the cross section of parts seen in FIG. 54 with two filters and one female/female connector affixed thereto for sterilizing matter communicated there into.

FIG. 56 is a cross section of the multi-chamber syringe seen in FIG. 54 with liquid communicated into a proximal chamber of the syringe and the cap, seen in FIG. 53, affixed thereto.

### DETAILED DESCRIPTION OF THE ILLUSTRATED EMBODIMENTS

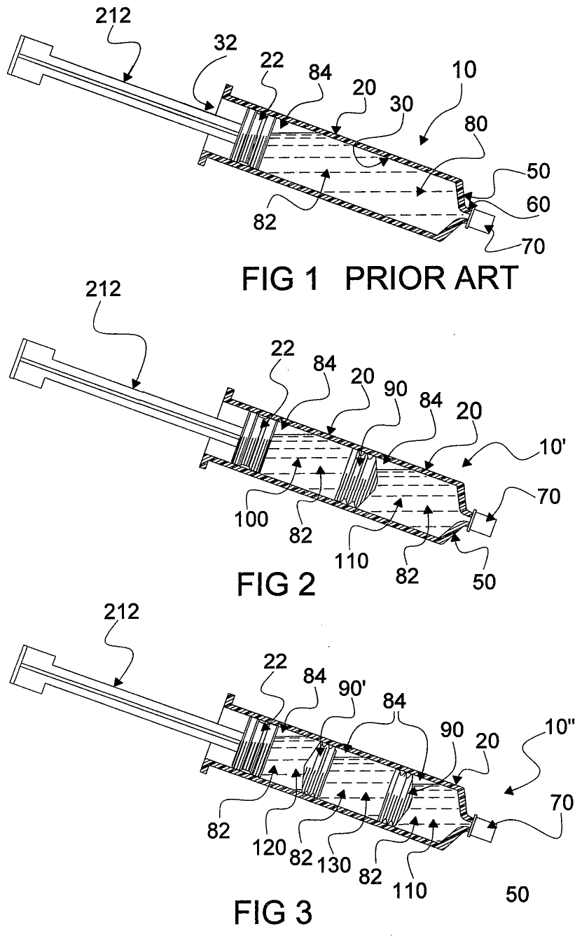

In this description, the term proximal is used to indicate nearness of a segment or portion of a device to a piston rod piston of a conventional medical syringe. The term distal refers the segment or portion being farther away therefrom. Reference is now made to the embodiments illustrated in FIGS. 1-46 wherein like numerals are used to designate like parts throughout. Primes of numbers identify parts which are similar, but not identical in form and function to numbered parts without primes.

Reference is now made to FIG. 1 wherein a conventional medical syringe 10 is seen to comprise a barrel 20 closed at one end by a piston rod piston 22 and therefrom comprising a substantially constant diameter inner surface 30 interrupted only by a proximal opening 32 where through the piston rod piston 22 has been displaced to reside within barrel 20 and a diminished distal section 50 where barrel 20 is narrowed to provide a fluid transmitting spout 60. Note that spout 60 is closed to fluid flow by a removable cap 70. As is common in contemporary medical syringe use, barrel 20 is seen to comprise but a single chamber filled with fluid comprising liquid 82 and a small pocket of gas 84. Gas 84 is normally air which was trapped during barrel filling of liquid 82.

FIG. 2 is similar to FIG. 1 except for a novel nested piston apparatus 90, being an example of the instant invention, which is disposed within barrel 20 to divide chamber 80 into two chambers, a proximal chamber 100 and a distal chamber 110, to provide a dual chamber syringe 10′. Note that each chamber contains fluid which comprises liquid 82 and gas 84. Different novel forms and functions of nested piston apparatuses are disclosed in detail hereafter. It is recognized that dual chamber syringes are known in medical syringe art, but none are known which employ nested pistons.

FIG. 3 is similar to FIG. 2 except for an additional novel nested piston apparatus 90′ also being disposed within barrel 20 to divide chamber 100 into two chambers, a more proximal chamber 120 and a medial chamber 130, thus forming a novel triple chamber syringe 10″. Note that each chamber contains fluid which comprises liquid 82 and gas 84. Of course, matter contained in each chamber 100, 110, 120 and 130 may be different in content and volume such that each chamber provides material for providing a specific medical function when dispensed from a syringe 10, 10′ or 10″.

Exemplary Application of a Nested Piston Apparatus for Providing a Sequentially Dispensing Dual Chamber Syringe 10′.

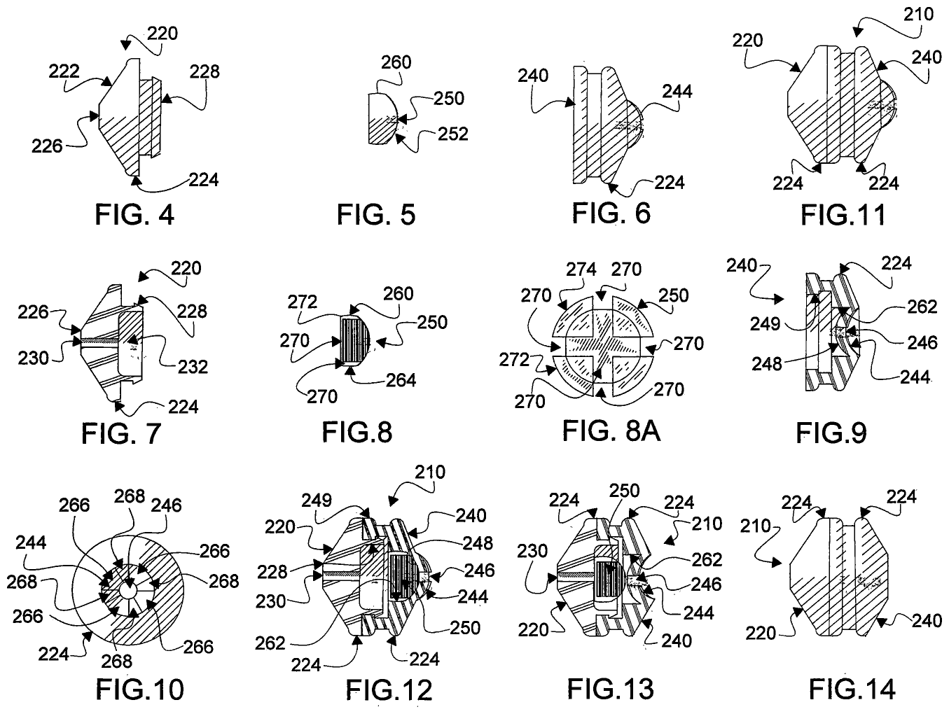

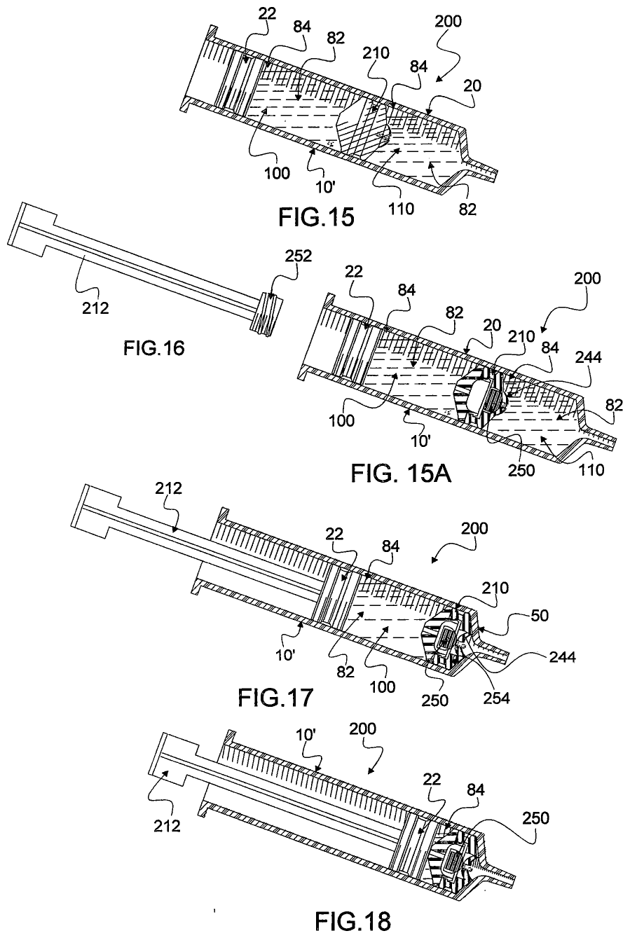

Reference is made to FIGS. 4-18 wherein parts and assemblies for a dual chamber syringe 10′ are seen. Such a dual chamber syringe 200 is seen in FIG. 15. A nested piston apparatus 210 is disposed in barrel 20 to provide two disparate chambers 100 and 110.

Note that a cap 70 is not shown for clarity of presentation. Also, note a plunger rod 212 (see FIG. 16) is not seen in FIG. Some automated medical device delivery machines are space limited. As disclosed hereafter, plunger rod 212 is preferably provided detachable to make storage in such machines and sites with limited capacity more reasonable.

Construction details of nested piston apparatus 210 are best seen in FIGS. 4-14. A proximally disposed piston 220 which is part of apparatus 210 is seen in FIG. 4. Piston 220 comprises a circular outer surface 222 which is enlarged to provide a wiping ring 224 distal from a reduced proximal nose 226. More distally located is a latch ring 228. As seen in FIG. 7, nose 226 comprises an entry orifice 230 for a liquid transmittal channel 232. When piston 220 is disposed in barrel 20, orifice 230 is disposed in a liquid only zone such that only liquid can flow there through and thus no potentially harmful gas can be dispensed to a patient line from a more proximal chamber.

A second piston 240 of apparatus 210 is seen in FIG. 6. Piston 240 is preferably molded as seen in FIG. 9 comprising at least one wiping ring 224 and a concave thin walled section 244 comprising a through hole 246 and an inner surface 248. In addition, piston 240 comprises a proximal catch ring 249. Piston 240 being made from sufficiently pliant material to permit section 244 to be displaced from a concave state to a convex state seen in FIG. 6. It is duly noted that each proximal and distal piston of a nested piston assembly comprises at least one wiping ring 224 as such may not be disclosed for each nested piston assembly described hereafter.

A part 250 which acts as a plug for hole 246 is seen in FIGS. 5 and 8. Plug 250 comprises a distal surface 252 designed to be in contact with section 244 and having a shape which is congruent to inner surface 248 when section 244 is disposed to be convex, as seen in FIG. 12 such that a hole 246 is made blind by plug 250 when disposed against inner surface 248. The importance of a blind hole such as blinded hole 246 is clarified by considering priming of a chamber distal to piston 240. Gas is facilely removed when priming such holes.

Nested piston apparatus 210 is assembled by displacing plug 250 into piston 240 and joining pistons 220 and 240 as seen in FIGS. 11 and 12. Note that latch ring 228 interlocks with catch ring 249 to form nested piston apparatus 210 (see FIG. 12).

So formed, apparatus 210 is disposed in barrel 20 as seen in FIG. 15A to provide chambers 100 and 110. Each comprises a volume of liquid 82. For displacement of liquid and pistons, a piston rod piston 212 comprises a threaded connector 252 which is designed to be affixed to piston 22 (connecting details not shown, but well known in medical syringe art).

When preparing for liquid delivery, chamber 110 is primed of air 84 by conventional syringe priming methods. Application of distally directed force upon plunger rod 212 displaces pistons 22 and 210 and liquid 82 thereby emptying chamber 110 as seen in FIG. 17. Contact of convex shaped section 244 with inner surface 254 of barrel section 50 inverts section 244, as seen in FIGS. 13 and 17.

The following valve related items which should be considered, for appropriate valve related action, at the time of apparatus 210 to barrel surface 254 impact are:
1. Hole 246 should remain closed until all fluid is dispensed from distal chamber 110.
2. Convex state of section 244 and position of plug 250 should remain as seen in FIG. 12 until such impact.
3. Continued contact after impact should invert section 244 and displace plug 250.
4. While section 244 is in contact with inner surface 254 of barrel 20 a dispensing pathway should be provided for draining fluid from chamber 110.
5. To reduce force required to invert section 244 and displace plug 250 proximally, plug 250 should have sidewall structure which is conducive to forcing plug proximally.
6. Plug 250 should be structured to provide pathways for liquid such that no fluid caused negative pressure resists plug 250 displacement.

When these considerations are met, section 244 inversion and plug displacement are permitted to occur without excessive force applied to plunger rod 212, a condition which permits use of dual chamber syringe 10′ in a conventional syringe pump.

To accomplish item 1 and 5, plug 250 comprises an arc shaped side surface 260 (see FIGS. 5 and 8) which is designed to snugly reside within channel 262 of piston 240. Peak 264 of surface 260 is disposed to coincide with the proximal end of channel 262 at a mid-point of inversion of section 244 such that displacement is resisted in early stages of plug 250 displacement but naturally accelerated thereafter.

To fulfill condition of item 3, continued distal displacement of apparatus 210 after impact fully inverts section 244 and displaces plug 250 as seen in FIGS. 13 and 14.

Consistent with requirement of item 4, as seen in FIGS. 10 and 11, section 244 has a surface structure comprising raised sections, generally numbered 266 adjoining lowered duct sections 268 such that pathways are provided for dispensing fluid from chamber 110 while section 244 is in contact with barrel inner surface 30 in diminished distal section 50 of barrel 20.

To satisfy conditions of item 6, plug 250 has a plurality of grooves, generally numbered 270, which are disposed in the proximal face 272 and cylindrical side 274 of plug 250 as seen in FIGS. 8 and 8A, grooves 270 being disposed to permit liquid to flow about plug 250 throughout displacement.

Noting, as seen in FIG. 13, plug 25 is sized and shaped to retain closure of hole 246 when proximally displaced as section 244 is inverted. However, when upstream pressure exceeds pressure of fluid distal from section 244, pliancy of section 244 permits sufficient displacement to provide a pathway for dispensing liquid from chamber 100 when force is applied to plunger rod 212, see result in FIG. 18. In this manner, liquid disposed in each chamber 100 and 110 is sequentially effectively displaced and delivered without dispensing gas from chamber 100.

Exemplary Application of a Nested Piston Apparatus for Providing a Sequentially Dispensing Dual Chamber 10′ Mixing Syringe 300.

Primary conditions for a dual chamber mixing syringe 10′ are as follows:
1. Matter in each of the two chambers should remain disparate until a valve is selectively opened to permit mixing.
2. Liquid disposed in a proximal chamber (a diluent) should be facilely displaced into a proximal chamber where mixing occurs.
3. Once liquid is so displaced into the distal chamber, the newly configured mixing syringe 10′ should operate in the same manner as a single chamber syringe 10.

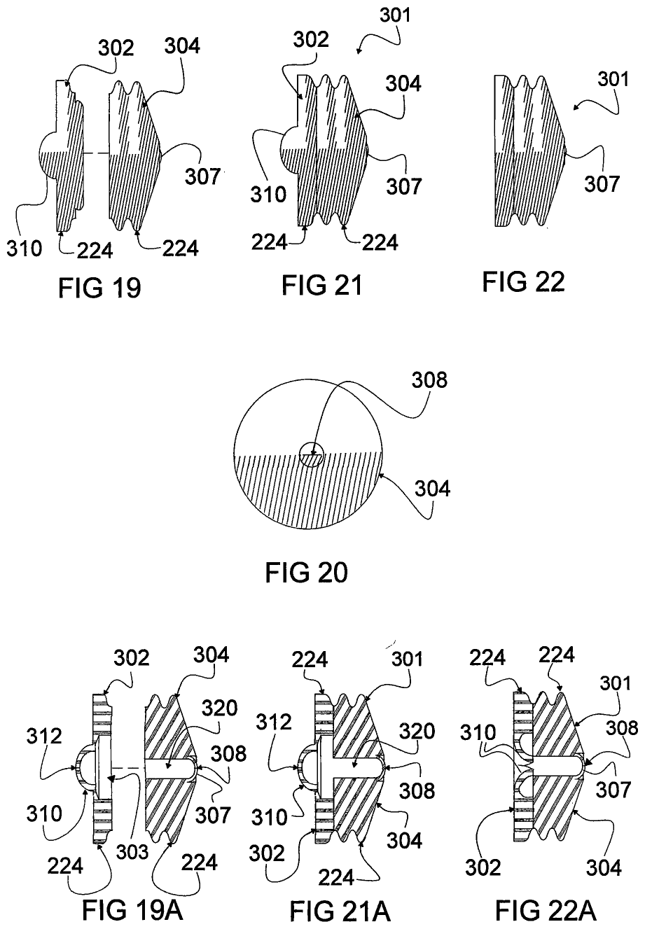

Reference is now made to FIGS. 19-25 wherein parts and assemblies for constructing a dual chamber syringe 10′ for providing a mixing syringe 300 are seen. As seen in FIGS. 19-22A, a nested piston apparatus 301 comprises two valved parts numbered 302 and 304.

As seen in FIGS. 21 and 21A, a nested piston apparatus 301 consists of a first valved piston 302 and a second valved piston 304 designed to be nested together to provide apparatus 301. Each valved part 302 and 304 comprises at least one wiping ring generally numbered 224.

It may be noted that no catch or latch rings are seen in the cited figures for joining parts 302 and 304 to form apparatus 301. As only two parts are joined to form nested piston apparatus there is no pre-syringe insertion requirement for such as, due to wiping ring 224 operation, such parts inserted together within a syringe will remain together when displaced therein by force of a pressure differential across the so assembled and nested piston apparatus.

As a further note, each wiping ring 224 interface with a syringe wall surface 30 is usually lubricated to reduce ring to wall friction. A tendency to wipe lubricant from space between a ring 224 can lead to reduction in lubrication in isolated ring 224/wall surface 30 interfaces which results in increased friction thereat causing a wiping ring 224 to “stick” (commonly called stiction) where lubricant has been reduced. Such selected stiction when displacing force is disposed against a free piston can cause the piston to tilt. Tilting can cause an interface between wiping ring 224 and surface 30 to part resulting in undesirable leakage across a ring 224. Likelihood of such occurring can be reduced by increasing free piston length (which is usually undesirable), thinning wiping rings, or providing material within a free piston which leaches additional lubrication upon application of a higher than normal applied differential displacement pressure.

As seen in FIGS. 19 and 19A, piston 302 comprises a pressure actuated valve in the form of an invertible dome 310 with a medially disposed slit 312. Further, piston 302 comprises an interiorly disposed empty chamber 303 as seen in FIG. 19A.

Also seen in FIGS. 19 and 19A is a piston 304 which comprises a medially disposed one-way valve 307 which is formed by a slit 308 (best seen in FIG. 20) to selectively close a chamber 320 within piston 304 and permit only distally directed fluid flow outward from inside apparatus 301. In assembly, pistons 302 and 304 are joined such that air (a compressible gas) is disparately trapped within apparatus 301. Pistons 302 and 304 are nested to form nested piston apparatus 301 as seen in FIGS. 21 and 21A.

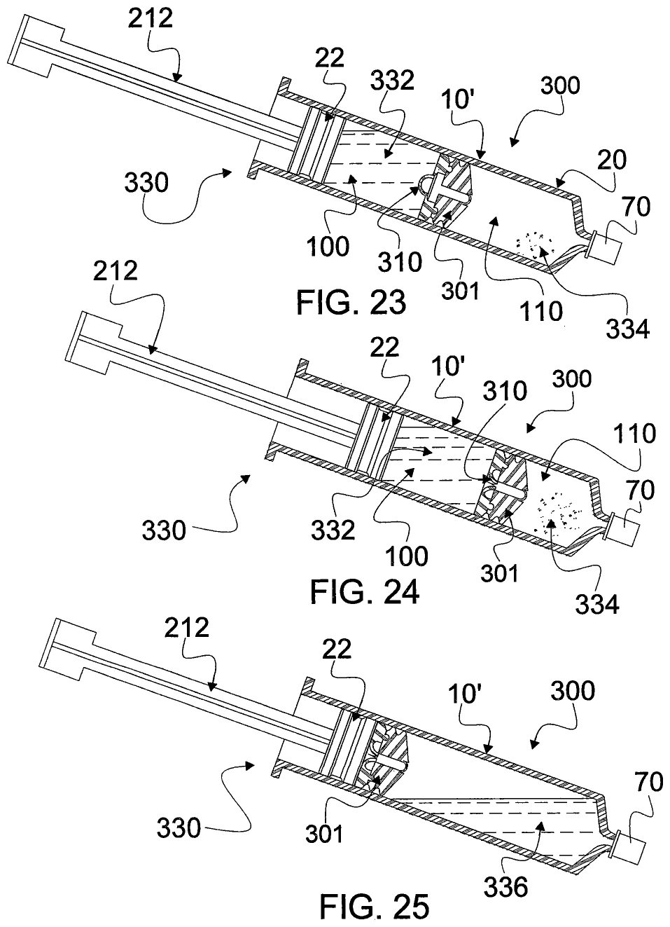

A dual chamber mixing syringe 330 is seen in FIG. 23 to comprise a proximal chamber 100 comprising a liquid 332 for mixing with matter 334 disposed in a distal chamber 110. Matter 334 may be solid (e.g. a lyophilized drug) or liquid.

Note that barrel 20 is proximally closed by a piston rod piston 22 and distally with a cap 70 such that displacement of plunger rod 212 toward cap 70 increases pressure within barrel 20 to increase pressure within chamber 110. So displaced, pressure exterior to nested piston apparatus 301 imposes a pressure differential across dome slit valve 310 which opens dome slit valve 310 as seen in FIGS. 24 and 22A. When dome slit valve 310 is so opened force on plunger rod 212 displaces liquid from chamber 100 into chamber 110, increasing pressure therein. Release of force from rod 212 permits increased pressure in chamber 110 to displace nested piston apparatus 301 proximally without displacement of fluid from distal chamber 110 proximally due to action of one-way valve formed by slit 308 (see FIG. 22A). Repeated application and release of force on rod 212 “pumps” liquid from chamber 100 into chamber 110 where liquid 332 and matter 334 are mixed to provide mixture 336 as seen in FIG. 25.

Exemplary Application of a Nested Piston Apparatus 301′ for Providing a Sequentially Dispensing, Diluent Sterilizing Dual Chamber Mixing Syringe 410.

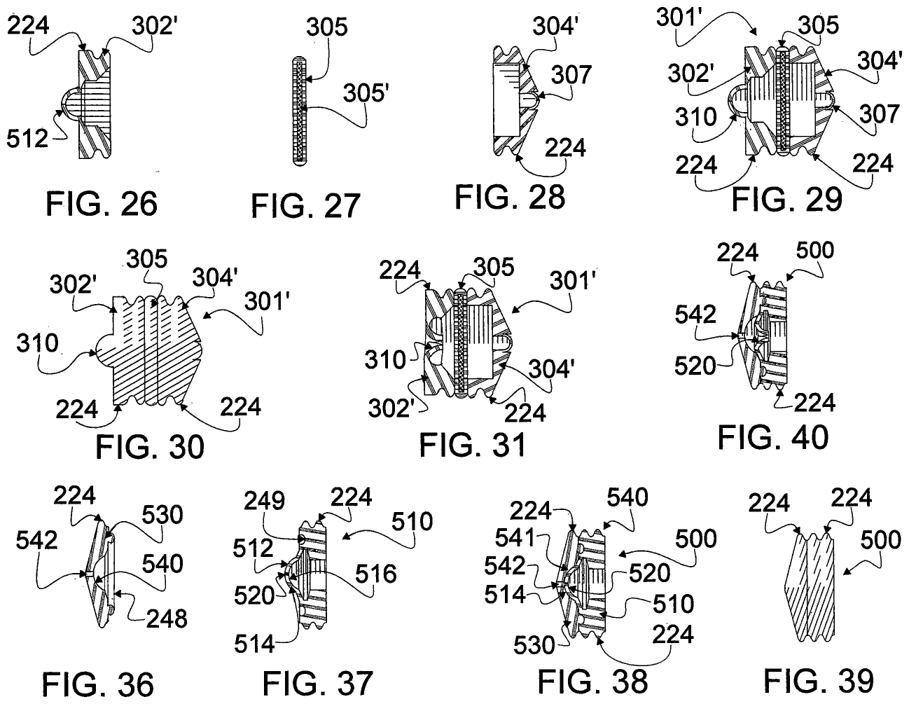

For those cases where it is desired to sterilize a diluent disposed in a mixing syringe prior to mixing, a filtering nested piston assembly 301′ seen in FIGS. 26-35 can be provided according to the instant invention.

Seen in FIG. 26 is a piston 302′ which is like piston 302 (seen in FIG. 19A without a latch ring which is not a necessary element in this application). Also, seen in FIG. 28 is a piston 304′ (which is like piston 304 without a catch ring which is also not a necessary element in this application). A filter piston 305 comprising a sterilization grade filter 305′ (e.g. a 0.2 micron filter) is seen in FIG. 27. A nested piston assembly 301′ comprising parts seen in FIGS. 26-28 is seen in FIGS. 29 and 30.

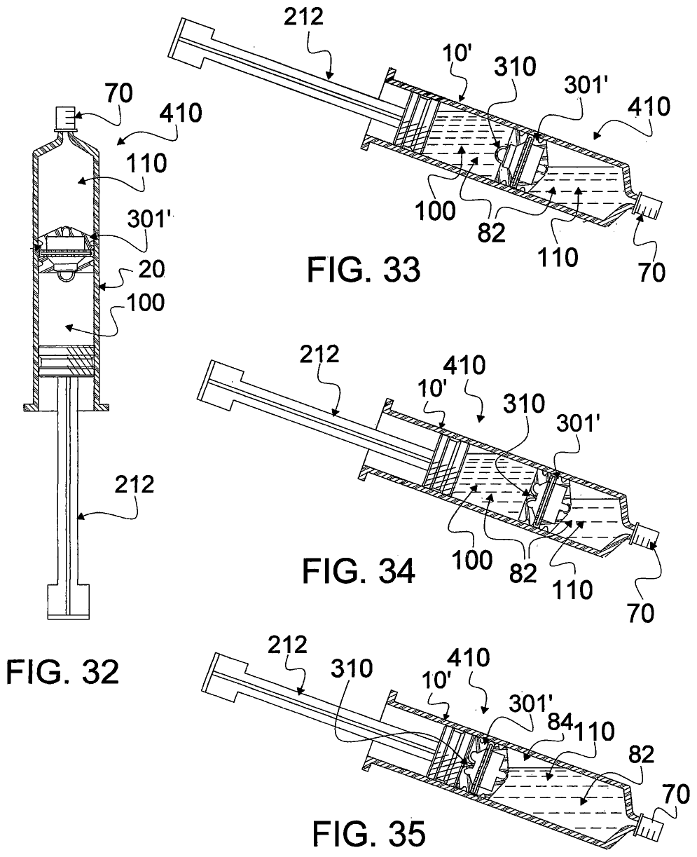

An empty filtering and mixing dual chamber syringe 410 having a nested piston assembly 301′ disposed within an upright syringe barrel 20 is seen in FIG. 32. Further, a filtered mixing syringe with filter 410 comprising liquid 82 in both chambers 100 and 110 is seen in FIG. 33. Application of force on plunger rod 212 operates to open a valve 310 as seen in FIGS. 34 and 35. Liquid 82 is pumped from chamber 100 into chamber 110 in the same manner liquid 82 is provided for mixing in mixing syringe 301 (see FIGS. 24 and 25) with a resulting mixture being provided in distal chamber 110 as seen in FIG. 35. To assure proper priming and assurance of no air block of fluid in nested piston assembly 301′, dispose syringe 410 upright as seen in FIG. 32 until first liquid 84 is communicated from chamber 100 into chamber 110.

Exemplary Application of a Nested Piston Apparatus for Providing a Triple Chamber Syringe.

This application example provides disclosure for a triple chamber mixing syringe, however, one who is skilled in multi-chamber syringe art would understand that commonly any dual chamber syringe can be fitted with a proximally disposed, nested piston apparatus as disclosed hereafter to provide an additional proximal chamber thereby providing a triple chamber syringe.

Reference is now made to FIGS. 36 to 46 wherein disclosure is provided for an exemplary triple chamber syringe 10″. Nested piston parts for a nested piston apparatus 500 are seen joined in FIG. 39, in cross section in FIG. 38 and apart in FIGS. 36 and 37.

A valved piston 510, comprising a pressure actuated dome valve 512, is seen in FIG. 37. Dome valve 512 comprises a convex dome 514 made of pliant material and a medially disposed slit 520 which is normally closed. Note that valve 512 is permissive to fluid flow from a concave side 516 of dome 514 when a pressure differential greater on concave side 516, is imposed. To assure that no fluid flows through valve 512 under such conditions, slit 520 must be restrained to keep dome valve 512 in a closed state.

To restrain dome valve 512 from undesired fluid transmission, a second piston 530 is joined with piston 510 to form a nested piston apparatus 500 as seen in FIG. 38. Piston 530 comprises a dome slit restraining section (a restrainer 541, see FIG. 38) which is sized and shaped to restrict slit 520 from parting. Restrainer 541 also comprises a hole 542 which permits fluid pressure external to restrainer 541 to engage dome 514. When piston 530 is nested with piston 510, slit 520 is held closed as seen in FIG. 38. However, when sufficient pressure to invert dome 514 is communicated through hole 542, dome 514 inverts opening slit 520 to a fluid flow permissive open state seen in FIG. 40.

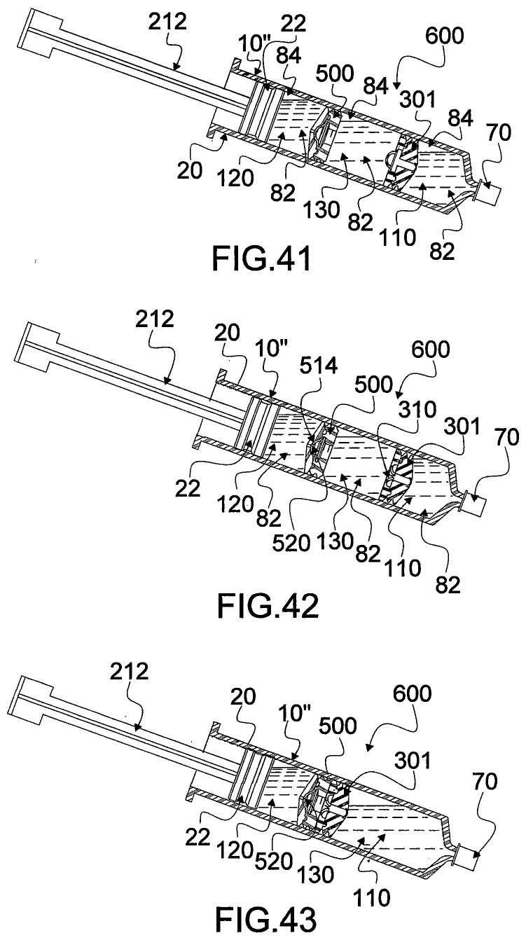

As seen in FIG. 41, a triple chamber mixing syringe 600 is made by displacing a nested piston apparatus 301 (see FIG. 21A) and a nested piston apparatus 500 into a syringe 10″ to provide chambers 120 and 130. Note that each of chambers 120, 130 and 110, in this example, is seen to contain liquid 82 and a small quantity of gas 84.

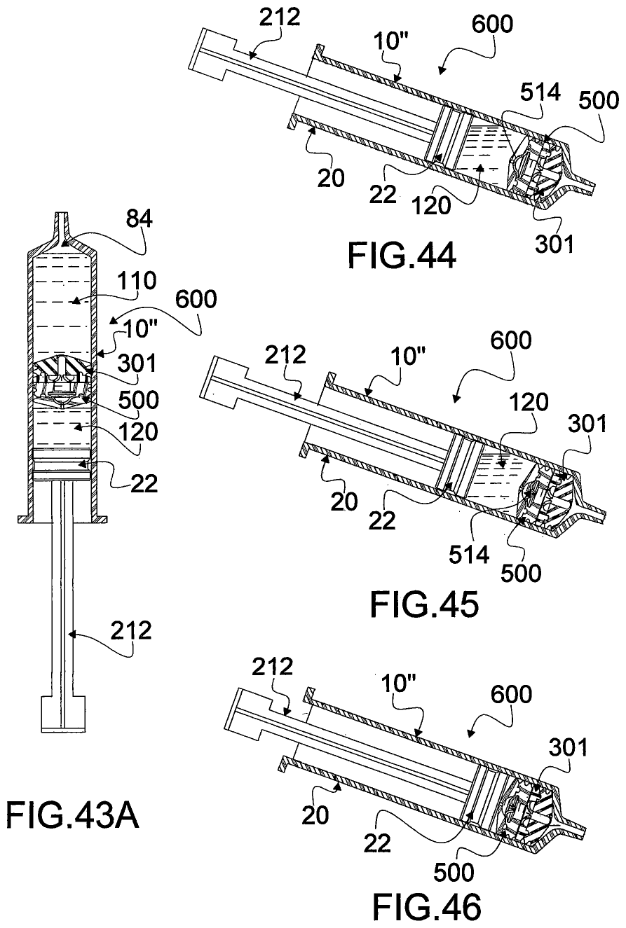

In FIG. 42, plunger rod 212 is displaced to force fluid within syringe 600 distally which results in opening valve 310 of apparatus 301 for pumping fluid to empty chamber 130 into chamber 110, as disclosed supra for a dual chamber mixing syringe 300 (see FIG. 23). Note that slit 520 remains closed as a pressure differential on both sides of dome 514 is not sufficient to invert dome 514 to an open state. Fluid from chamber 130 is seen displaced into chamber 110 for mixing in FIG. 43.

Syringe 600, with cap 70 removed is seen in FIG. 43A disposed for priming gas from chamber 110. In FIG. 44, plunger rod 212 is displaced to empty chamber 130. Continued displacement of plunger rod 212 applies sufficient differential pressure to invert dome 514 to an open state as seen in FIG. 45. Further displacement of plunger rod 212 empties chamber 120 as seen in FIG. 46. In this manner, all fluid in each chamber of syringe 600 is kept disparate until sequentially and selectively displaced by simple forcing action upon plunger rod 212.

Exemplary Application of a Dual-Chamber Syringe Apparatus Whereby the Syringe Apparatus is Provided Dry (Fully Assembled, but Unfilled) to a User Site Before being Filled Thereat for Use.

Filling one or more closed chambers of a dual-chamber syringe, in controlled and sterile environments is well known in the medical syringe art. However, it is believed that communicating liquid to a back chamber of a dual-chamber syringe within a potentially contaminating environment has not been taught.

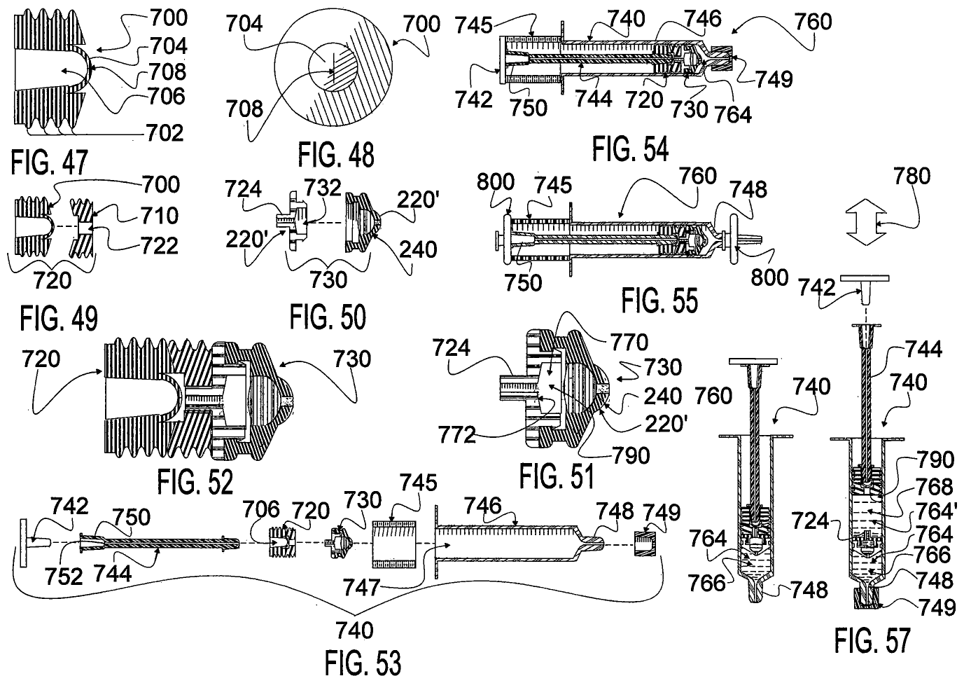

Reference is now made to FIGS. 47-57 wherein apparatus and methods for accomplishing filling each of a back or proximal chamber and a front or distal chamber, such that both can be filled while retaining sterility in a potentially contaminating environment is disclosed. A piston used for filling a back chamber of a dual-chamber syringe is seen in FIGS. 47 and 48. As seen in FIG. 47, a piston 700 is seen to comprise a plurality of wiping rings, commonly numbered 702, and a dome shaped closure 704 of an open cavity 706. Closure 704 comprises a slit 708, better seen in FIG. 48. Shape of closure 704 and corresponding slit 708 act jointly to provide a one-way valve permissive to fluid flow from cavity 706, as one skilled duck-bill valves understands.

A spacing piston 710 is seen in FIG. 49 where pistons 700 and 710 are seen as a prospective nested pair 720. Piston 710 is sized and shaped to be nested with piston 700 and provides an open hole 722 which provides a pathway for dispensing liquid there through and also provides an enclosure for an extended tube 724 (see FIGS. 50 and 51) which is used for communicating with a liquid only zone and reduces space for resident gas when provided dry.

A second prospective pair 730 of nested pistons is seen in FIG. 50. Pair 730 consists of a piston 220′ and piston 240 (see FIGS. 7 and 12, as disclosed supra). Piston 220′ is similar in shape and function to piston 220, but provides a funnel shaped cavity 732 whereby gas is facilely pumped therefrom. Pair 730 is seen joined in FIG. 51.

Nested pairs 720 and 730 are seen nested together in FIG. 52. Parts of a so-fillable dual-chamber syringe assembly 740 are seen in FIG. 53 to comprise a plunger rod cap 742, a fluid communicating plunger rod straw 744, a nested piston pair 720, another nested piston pair 730, a removable guard 745 for restricting depth of penetration of pair 730 into a conventional syringe barrel 746, having an open end 747 and a fluid communication snout 748 (which is preferably shaped as a luer fitting), and a barrel cap 749.

Note that straw 744 has a proximally disposed female fitting 750, preferably being a luer or luer-lock connection. Fitting 750, is o[en to a small diameter pathway 752, at a distal end of which is a male fitting 754 size and shape of which is complimentary to cavity 706 such that insertion of fitting 754 into cavity 706 provides a fluid-tight connection. Fitting 754 may also be affixed to cavity 706 via barb impediments (not shown) molded onto fitting 754 for providing a connection which can be used a conventional plunger rod for filling and dispensing fluid from a syringe barrel.

A dry assembly 760, consisting of parts seen in FIG. 53, is seen in FIG. 54. As manufactured, with a cap 742 affixed to fitting 750, assembly 750 is preferably sterilized and provided dry within a protective cover, as is common for contemporary syringes. For this reason, cap 742 must be removed before chamber 762 is filled.

Due to piston valve 220′ being designed to actuate to an open state upon collision with the diminished distal barrel section 50, as seen in FIG. 17, piston 700 must be kept disparate from barrel section 50 until front or distal chamber 764 is filled with an incompressible fluid (e.g. liquid medicine) to assure no inadvertent actuation. It is for this purpose that a removable guard 745 is disposed between cap 742 and barrel 746 (see FIG. 54).

Chamber 764 is filled with liquid 766 by removing cap 749 from spout 748 (see FIG. 53) and drawing liquid 766 into chamber 764 by conventional syringe use technique. Note that, once chamber 764 is filled, the valve of piston 700 cannot be actuated to an open state until chamber 764 is emptied. At this point, cap 749 is affixed in fluid-tight relationship with snout 748 as seen in FIG. 57.

By dispensing liquid through straw 744, to breach nested pair 720 via slit 708 and, thereby, separate pairs 720 and 730, a liquid 764′ filled chamber 768 is formed, as seen in FIG. 57. As is well known in the medication dispensing art, no gas should be resident in delivered medication. As seen in FIG. 51, nested piston 730 has a cavity 770 which is gas 790 filled when assembly is delivered dry. Note that cavity 770 is in direct communication with extended tube 724 and has a funnel shaped structure 772 which is permissive to fluid communication through extended tube 724. Once chamber 762 is filled with liquid 764, piston rod cap 742 can be affixed to piston rod 744 and gas, then, can be pumped from cavity 770 by rotating filled assembly 760 vertically (as seen in FIG. 57) such that snout 748 is “down” and extended tube 724 is “up”. Then, successively applying downward force upon cap 742, as indicated by arrow 780, gas is pumped from cavity 770 by releasing gas 790 through extended tube 724 into chamber 768 in upward direction of arrow 780. Thus, by continuing to apply force and then releasing pressure upon cap 742, until no gas bubbles are seen to be delivered into chamber 762, cavity 762 is purged of gas 790. With all gas 790 communicated into chamber 768, whereby only liquid can be communicated from chamber 768, as extended tube 724 only communicates there after with a liquid only zone within chamber 768, no gas is dispensed from cavity 770.

In some cases, it is desirable to sterilize liquid being communicated into chambers 768 and 764. For this purpose each of two filters, commonly numbered 800, can be fitted to fitting 752 and snout 748 as seen in FIG. 55.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed and desired to be secured by Letters Patent is:

1. A nested piston apparatus system comprising:
a conventional medical syringe comprising a piston rod, a piston rod piston and an associated elongated barrel; and
at least one nested piston apparatus disposed within said barrel, each said apparatus comprising a plurality of pistons nested together to thereby comprise a single free piston which when disposed in said barrel divides a single chamber within said barrel into a proximal and a distal chamber, each of said pistons comprising structure which is not conducive to being formed as part of another piston within the apparatus;
each said apparatus comprising a distal piston and a proximal piston wherein each of said distal and proximal pistons comprise at least one wiping ring;
at least one of said plurality of pistons comprising a valve which is normally closed such that fluid within the barrel distal from the free piston is kept disparate from fluid proximal from the free piston and is selectively opened to be permissive to fluid flow from a more proximal chamber.

2. A nested piston apparatus system according to claim 1 wherein said barrel is diminished in diameter at a distal end to form a dispensing spout which comprises a fluid pathway formed by a through hole and said nested piston apparatus comprises a first piston comprising a normally closed valve which is opened upon impact with the diminished end of the barrel and a second piston which comprises a filtering component which passes liquid but obviates passage of gas whereby insertion of said nested piston apparatus comprising said valve and said filtering component into said barrel to divide a single chamber into a distal and a proximal chamber provides a dual chamber sequential delivery syringe which permits priming of the distal chamber by conventional syringe priming technique and sequential dispensing of fluid from only the distal chamber then, after opening of the valve, from the proximal chamber without dispensing air from the proximal chamber.

3. A nested piston apparatus system according to claim 2 wherein said filtering component comprises a single distal pathway from a liquid only zone disposed in the proximal chamber through said valve, when opened, to assure only liquid being dispensed from the proximal chamber.

4. A nested piston apparatus system according to claim 1 wherein said nested piston apparatus comprises a distally disposed piston comprising a one-way valve which is permissive to distal flow and another proximally disposed piston comprising a pressure actuated, normally closed valve, said pistons being nested together to form a hollow housing resident there between which provides a pathway for fluid flow from said proximal chamber into said distal chamber when said valve is open; said valve being actuated to an open state by a pressure differential disposed, across the pressure actuated valve resulting from an application of force applied to said piston rod; said proximal chamber comprising a bolus of compressible fluid such that successive applications of force via said piston rod, when said pressure actuated valve is open, dispenses liquid through said one-way valve into the distal chamber from the proximal chamber thereby increasing pressure in the distal chamber which displaces the free piston proximally when force is relieved from said piston rod and whereby content of the proximal chamber is sequentially displaced into the distal chamber for mixing.

5. A nested piston apparatus system according to claim 4 wherein said pressure actuated valve is a dome valve.

6. A nested piston apparatus system according to claim 4 wherein said said nested piston apparatus comprises a medially disposed third piston between the distal and proximal pistons, said third piston comprising a sterilizing filter through which filters all fluid being displaced between the proximal and distal pistons to thereby assure contamination of fluid in said proximal chamber is not communicated to matter in the distal chamber.

7. A nested piston apparatus system according to claim 2 wherein said at least one nested piston apparatus comprises two free pistons disposed within said barrel to provide a combination comprising a chamber distal to a first free piston, a chamber medially disposed between the first free piston and a second free piston and a chamber proximally disposed relative to the second free piston, each free piston comprising a proximally disposed piston comprising a filter to retard passage of gas there through nested with a piston comprising a normally closed valve, to assure disparity between chambers, said valve of said first free piston being opened by collision of the first piston and the diminished end of said barrel and said valve of second free piston being opened by a pressure differential which occurs when said second free piston is displaced into contact with said first free piston such that contents of each of said chambers are separately and selectively dispensed as follows:
a. content of the distal chamber is dispensed while valves of said first and said second free piston are closed;
b. content of the medial chamber is dispensed after said valve of said first free piston is opened by impact of collision of first free piston with the diminished end of said syringe;
c. content of the proximal chamber is dispensed after said valve of said second free piston is opened by a pressure differential occurring after the second free piston is immobilized by contact with said first free piston to thereby provide for disparate dispensing of contents of each chamber of a three chamber sequential delivery syringe.

8. A nested piston apparatus system according to claim 4 further comprising a second nested piston apparatus comprising a normally closed, pressure actuated valve and a filter which is permissive to liquid flow but impervious to gas flow to provide a second free piston which is disposed in said distal chamber to create a combination medial chamber and a newly formed distal chamber, contents of the two chambers being disparate while the pressure actuated valve is closed, said pressure actuating valve being opened as a result of impact between said two free pistons, said medial chamber comprising a diluent for mixing and said newly formed distal chamber comprising a flush liquid, thereby providing a mixing syringe with flush.

9. A nested piston apparatus system according to claim 2 wherein said normally closed valve comprises a sensing element integrally disposed as a most distal part of said first piston, said piston comprising pliant material and an open medial channel providing a fluid pathway through said valve when opened and said element comprising an as molded concave shape which is wholly disposed within said channel and an open medially disposed hole, said element being formed to have two stable states, a first state being the concave molded shape when said element resides within said channel and a second state being convex when the element is displaced distally to provide a bulbous distally disposed barrel sensing surface disposed to impact said barrel before any other portion of said piston, and a plug which is sized and shaped to fit snugly within said channel and close said hole such that upon impact said element is reverted to the first state with the plug initially retaining hole closure such that said valve is opened, to permit fluid flow through said channel, by pressure resulting from force applied to said piston rod after the element is reverted.

10. A nested piston apparatus system according to claim 2 where-in said normally closed valve comprises a fluid dispensing hole which is closed by a plug which provides a closure for said hole when said plug is in contact with said hole, said plug remaining in contact with said hole during valve opening to assure dead space between said barrel and said nested piston apparatus is independent of valve opening dynamics.

11. A nested piston dual-chamber piston apparatus which is provided unfilled and dry to a user, each such chamber being maintained in a sterile state while being filled with a liquid medication within a potentially contaminating environment for later use as a syringe which dispenses only liquids.
